(19) United States
(12) Patent Application Publication
(10) Pub. No.: US 2023/0357174 A1
(43) Pub. Date: Nov. 9, 2023

(54) TALL OIL DERIVED GLYCIDYL ESTERS AND PROCESS OF MAKING THE SAME

(71) Applicant: INGEVITY SOUTH CAROLINA, LLC, North Charleston, SC (US)

(72) Inventors: Bing Wang, Mount Pleasant, SC (US); Mitra Shiran Ganewatta, Ladson, SC (US)

(21) Appl. No.: 17/906,706

(22) PCT Filed: Mar. 17, 2021
(86) PCT No.: PCT/US2021/022669

§ 371 Date: Sep. 19, 2022

Related U.S. Application Data
(60) Provisional application No. 62/992,461, filed on Mar. 20, 2020.

(51) Int. Cl. C07D 301/30

(52) U.S. Cl. CPC C07D 301/30

(57) ABSTRACT

Presently described are methods for preparing glycidyl esters. The methods described herein provide quantitative conversion of carboxylic acid substrates to halohydrin intermediates using a small excess of epihalogenhydrin and performing the ring-closing step at a temperature of up to 30° C. unexpectedly reduce the formation of side-products in the ring-closing step. The described methods are also applicable to rosin derivatives and fatty acid derivatives. Utilizing these glycidyl esters as raw material, glycidyl ester derivatives with improved purity can be made.

### CROSS REFERENCE TO RELATED APPLICATIONS

This application claims the benefit of U.S. Application No. 62/992,461, filed Mar. 20, 2020, which is incorporated herein by reference in its entirety.

### BACKGROUND

### 1 Field of the Discovery

The present disclosure relates to methods to prepare glycidyl esters of rosin acids, fatty acids, and derivatives thereof. In certain aspects the rosin acids, fatty acids, and derivatives thereof are derived from tall oil rosins, gum rosins or wood rosins. The disclosure also provides methods of making derivatives from these glycidyl esters.

### 2 Background Information

Thermosetting polymers, such as epoxy resins, have been widely used in in coatings, adhesives and composite materials. Historically, epoxy resins have been synthesized using petroleum-based chemicals as raw materials. However, due to increasing environmental concerns, there is a need for epoxy resins derived from bio-renewable raw materials.

Rosin, a bio-renewable raw material, is commercially available, and can be obtained from pine trees by distillation of oleoresin (gum rosin being the residue of distillation), by extraction of pine stumps (wood rosin) or by fractionation of tall oil (tall oil rosin). Rosin contains a mixture of rosin acids, fatty acids, and other unsaponifiable compounds.

Tall oil, a type of rosin (originating from the Swedish word “tallolja” (“pine oil”)) is obtained as a by-product of Kraft pulping in the paper making process. A product of the Kraft process, crude tall oil (CTO), can be further purified by distillation to provide tall oil heads, tall oil fatty acids (TOFA), distilled tall oil (DTO), tall oil rosin (TOR), and tall oil pitch. These products have long been used in traditional fields such as inks, adhesives, oil fields, mining, paper sizing and detergents.

As described in U.S. Pat. No. 2,893,875, a conventional method for preparing glycidyl esters from carboxylic acids utilizes an alkali metal soap of the carboxylic acid, followed by reaction with epichlorohydrin to provide the glycidyl ester of the carboxylic acid in a single step. This reaction is typically biphasic, with the soap in the aqueous phase and the acid in the organic phase. In addition to the glycidyl ester product, this approach results in side-products, such as ring-opened epoxides and dimers (e.g., diglycerides). Therefore, the % yield of the glycidyl ester product obtained using this method is low and the glycidyl ester product is impure due to contamination with undesirable side-products.

Another method for preparing glycidyl esters includes two steps: reaction of the carboxylic acid with epichlorohydrin to provide a ring-opened (i.e., halohydrin) intermediate; followed by ring-closing reaction to provide the glycidyl ester product. The ring-closing step using this conventional method is complicated by the production of side-products, particularly in the case of primary carboxylic acids such as fatty acids. Some literature procedures attempt to overcome this problem of side-product formation by performing the ring-closing reaction as a biphasic reaction under basic conditions. In this approach, the halohydrin intermediate is in the organic phase and in close contact with the aqueous base, and thus prone to be hydrolyzed due to higher reaction temperature required (normally up to 80° C.). As such, the ring-closing reaction results in a significant amount of hydrolysis side-product (i.e., soap), which complicates phase separation and isolation of the glycidyl ester.

Some methods have attempted to remove the water produced during the second step by the addition of CaO. This method is ineffective due to sluggish liquid/solid interaction, but it also complicates the subsequent separation of spent caustic solid from the glycidyl ester product.

While conventional methods can be used to prepare glycidyl esters from carboxylic acids, they suffer from several key disadvantages, including lackluster yields, the formation of undesirable side-products, and being overly cumbersome and therefore not amenable to scale-up. As such, there is a need for improved methods for the preparation of glycidyl esters from carboxylic acid substrates that are amenable to scale-up procedures and therefore suitable for industrial applications.

### SUMMARY

Presently described are methods for preparing glycidyl esters of rosin acids, fatty acids and their derivatives thereof. The methods described herein surprisingly and unexpectedly provide the glycidyl esters in good yield and purity. Utilizing the described glycidyl esters as raw materials, derivatives of rosin acids glycidyl esters and fatty acid glycidyl esters can be made with improved purity.

In any of the aspects or embodiments described herein, a method is disclosed for the preparation of a glycidyl ester comprising the steps of
a. admixing a carboxylic acid substrate comprising a rosin acid or derivative thereof, a fatty acid or derivative thereof, or a combination thereof; a catalyst; a molar excess of an epihalogenhydrin based on the total moles of carboxylic acid groups; and optionally an organic solvent to form a reaction mixture;
b. heating the reaction mixture at a temperature from about 60 to about 125° C. to form a halohydrin intermediate;
c. combining the reaction mixture comprising the halohydrin intermediate from step (b) with a basic solution comprising an alkaline base, water, optionally, a water-soluble organic solvent; and optionally an organic solvent to form a reaction mixture; and
d. allowing the reaction to proceed at a temperature of up to about 30° C. to provide the glycidyl ester.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure can be utilized in numerous combinations, all of which are expressly contemplated by the present disclosure. These additional advantages objects and embodiments are expressly included within the scope of the present disclosure. The publications and other materials used herein to illuminate the background of the invention, and in particular cases, to provide additional details respecting the practice, are incorporated by reference.

### DETAILED DESCRIPTION

The present disclosure will now be described more fully hereinafter, but not all embodiments of the disclosure are shown. While the disclosure has been described with reference to exemplary embodiments, it will be understood by those skilled in the art that various changes can be made and equivalents can be substituted for elements thereof without departing from the scope of the disclosure. In addition, many modifications can be made to adapt a particular structure or material to the teachings of the disclosure without departing from the essential scope thereof.

Where a range of values is provided, it is understood that each intervening value between the upper and lower limit of that range and any other stated or intervening value in that stated range is encompassed within the invention. The upper and lower limits of these smaller ranges can independently be included in the smaller ranges is also encompassed within the invention, subject to any specifically excluded limit in the stated range. Where the stated range includes one or both of the limits, ranges excluding either both of those included limits are also included in the present disclosure.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of’ and “consisting essentially of’ shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

### Exemplary Aspects and Embodiments

Surprisingly and unexpectedly, the inventors discovered that glycidyl esters of rosin acids, fatty acids, or derivatives thereof could be prepared in a one-pot reaction by reacting the carboxylic acid group(s) of the carboxylic acid substrate with a small excess of epihalogenhydrin optionally, in an organic solvent to form a halohydrin intermediate, followed by a ring-closing reaction at a temperature of up to about 30° C. to provide a glycidyl ester, while decreasing the amount of side-product formation. It was particularly surprising that the ring-closing reaction, which was carried out at lower temperatures than the temperatures used in conventional ring-closing methods, was typically complete in about 8 hours or less. In some embodiments, the reaction time is less than about 8 h, less than about 6 h, less than about 4 h, less than about 2 h, less than about 1 h, or less than about 0.5 h, depending on the temperature of the reaction and the and rate of addition of the basic solution. Using conventional methods, the ring-closing step typically takes about 24 h with heating and is accompanied by significant side-product formation (15-25%). Therefore, the ring-closing step of the disclosed methods proceed faster than conventional methods at lower temperatures and with a lower amount of undesirable side-products formed. The disclosed methods relate to the preparation of glycidyl esters from rosin acids, fatty acids, and derivatives thereof; the glycidyl esters of rosin acids, fatty acids, and derivatives thereof obtained using the disclosed methods; the preparation of derivatives from the glycidyl esters; the derivatives of the glycidyl esters obtained using the disclosed methods; products derived from the glycidyl esters; and products derived from derivatives of the glycidyl esters.

As described above, prior methods for the preparation of glycidyl esters from carboxylic acid substrates suffer from well-known disadvantages that prevent them from implementation, including not being scalable, not being economically feasible, having a low yield and/or purity due to side-product formation, and/or involving waste stream management issues.

Thus, in an aspect, the description provides a method comprising the steps of (a) admixing a carboxylic acid substrate comprising a rosin acid or derivative thereof, a fatty acid or derivative thereof, or a combination thereof; a catalyst; a molar excess of an epihalogenhydrin based on the total moles of carboxylic acid groups; and optionally an organic solvent to form a reaction mixture; (b) heating the reaction mixture at a temperature from about 60 to about 125° C. to form a halohydrin intermediate; (c) combining the reaction mixture comprising the halohydrin intermediate from step (b) with a basic solution comprising an alkaline base, water, optionally, a water-soluble organic solvent; and optionally an organic solvent to form a reaction mixture; and (d) allowing the reaction to proceed at a temperature of up to about 30° C. to provide the glycidyl ester.

Advantageously, the methods described herein provide glycidyl esters in good yield and in good purity in a shorter total reaction time.

In any of the aspects or embodiments described herein, the carboxylic acid substrate includes rosin acids, or derivatives thereof, fatty acids, or derivatives thereof, or a combination thereof.

Rosin acids include C₂₀ mono-carboxylic acids with a core having a fused carbocyclic ring system comprising double bonds that vary in number and location. Examples of rosin acids include abietic acid, neoabietic acid, pimaric acid, levopimaric acid, sandaracopimaric acid, isopimaric acid, and palustric acid. TOR can further contain dimerized rosin acids and dehydroabietic acids formed during the Kraft process and distillation of CTO.

TOFA includes a complex mixture of fatty acids, including, e.g., palmitic, stearic, oleic, elaidic, and linoleic acids.

The disclosed methods can be used to prepare glycidyl esters of rosin acid derivatives and fatty acid derivatives. Rosin acid derivatives and fatty acid derivatives can include Diels-Alder adducts. Diels-Alder cycloaddition can be used to form what are commonly called “rosin adducts” from rosin acids and “fatty acid adducts” from fatty acids. Diels-Alder adduction occurs with s-cis conjugated double bonds, or double bonds capable achieving a conjugated s-cis configuration. For example, abietic-type rosin acids undergo Diels-Alder adduction. Among the fatty acids present in tall oil products, oleic acid, linoleic acid, linolenic acid have double bonds capable of undergoing an ene reaction (as is the case for oleic acid because it has a single double bond) or Diels-Alder cycloaddition (for linoleic acid and linolenic acid).

Non-limiting exemplary dienophiles that can be used to react with conjugated dienes include maleic anhydride, fumaric acid, acrylonitrile, itaconic anhydride, and acrylic acid. Diels-Alder products obtained from the reaction of maleic anhydride with a rosin acid or a fatty acid have three carboxylic acid groups and are referred to as “maleated rosin” and “maleated fatty acid,” respectively. Similarly, Diels-Alder products obtained from the reaction of fumaric acid with a rosin acid or a fatty acid have three carboxylic acid groups and are referred to as “fumarated rosin” and “fumarated fatty acid,” respectively.

Rosin acid derivatives and fatty acid derivatives include dimers. The double bonds of rosin acids can react with each other to form rosin dimers. Similarly, the double bonds of fatty acids can react with each other to form fatty acid dimers. Rosin dimer molecule is a C₄₀-terpene typically having two double bonds and two carboxylic acid groups. Rosin dimerization can be controlled to obtain appropriate levels of dimerization; hence the dimer rosin product may be a mixture of rosin and dimerized-rosin molecules.

Rosin acid derivatives include hydrogenation products. Because the unsaturated double bonds of rosin acids are prone to oxidation and cause discoloration of a product, it may be desirable to reduce the probability of oxidation by reducing the number of double bonds in a rosin acid. Rosin acids can be partially hydrogenated to saturate one of the double bonds of the rosin acid or fully hydrogenated to saturate both double bonds.

Rosin acid derivatives include dehydrogenation products, also referred to as disproportionation products. This process can be used to reduce the conjugated double bonds in some rosin acids, making the resulting disproportionated rosin less susceptible to oxidation. The reaction takes places between the dienes of two identical rosin acids, where one is hydrogenated and the other is dehydrogenated, thus altering the ratios of the rosin acids from the untreated rosin. Similarly, fatty acid derivatives can include disproportionation products (e.g., oleic acid).

Derivatives of rosin acid and fatty acids include carboxylic acid salts. Salts include salts of rosin acids or fatty acids having monovalent cations (“soaps”) and salts of rosin acids having divalent cations (“rosinates”).

Derivatives of rosin acid and fatty acids include oxidized rosin acids and oxidized fatty acids. The double bonds of rosin acids and fatty acids are prone to isomerization and oxidation when exposed to heat, air, light, and mineral acids thus providing a mixture oxidation products.

The carboxylic acid substrates can be derived from wood rosin, gum rosin, or tall oil rosin. In some embodiments, the fatty acid is derived from at least one of a plant oil, crude tall oil, tall oil fatty acid, distilled tall oil, coconut oil, palm oil, rosin, tall oil rosin, gum tree rosin, wood rosin, softwood rosin, hardwood rosin, derivatives thereof, or a combination thereof. In some embodiments, the rosin acid is derived from crude tall oil, rosin, tall oil rosin, gum tree rosin, wood rosin, softwood rosin, hardwood rosin, distilled tall oil, derivatives thereof, or a combination thereof.

The carboxylic acid substrates can include a combination of rosin acids or derivatives thereof and fatty acids or derivatives thereof. For example, one skilled in the art appreciates that commercial TOFA contains some TOR, and commercial TOR also contains various levels of TOFA. In such embodiments, the carboxylic acid substrates are derived from crude tall oil, tall oil fatty acid, distilled tall oil, tall oil rosin, gum tree rosin, wood rosin, softwood rosin, hardwood rosin, a natural oil, or a combination thereof. The natural oil can include vegetable oil, safflower oil, sesame oil, canola oil, olive oil, oil, coconut oil, or a combination thereof.

In the disclosed methods, the methods include a first step of forming a reaction mixture from a carboxylic acid substrate. The reaction can be performed neat or in the presence of organic solvent. The organic solvent, when present, can include toluene, xylene, hexane, heptane, and mixtures thereof. In the first step, a molar excess of epihalogenhydrin is used. In conventional methods of preparing glycidyl esters, a very large molar excess of epihalogenhydrin can be used (e.g., 10 or more equivalents (eq.)), thus requiring the removal of the unreacted epihalogenhydrin from the reaction mixture, prior to the ring-closing step or complicating the work-up of the glycidyl ester. The disclosed methods utilize a molar excess of less than about 3 equivalents (eq.), less than about 2.5 eq., less than about 2.0 eq., from greater than about 1 to about 1.5 eq., from greater than about 1 to about 1.4 eq., from greater than about 1 to about 1.3 eq., from greater than about 1 to about 1.25 eq., or from greater than about 1 to about 1.2 eq., each based on the equivalents of carboxylic acid groups in the carboxylic acid substrate. Advantageously, removal/recovery of unreacted epihalogenhydrin from the reaction mixture is not required using the disclosed methods because a small excess is sufficient to provide complete conversion of the carboxylic acid substrate to the halohydrin intermediate. This improvement makes the methods amenable to scale-up, as the small excess of epihalogenhydrin can be hydrolyzed to glycerol, removed during the work-up, and does not cause phase-separation difficulties. There is no need to implement an epihalogenhydrin recovering step, which is costly and creates environmental concerns.

A catalyst is present during the formation of the halohydrin intermediate of the first step of the disclosed methods. The catalyst can include onium salts with various alkyl chains. Non-limiting exemplary catalysts include quaternary ammonium salts, phosphines, or phosphonium salts. Specifically, the catalyst can include tetrabutyl ammonium halides, such as tetrabutyl ammonium bromide, triphenylphosphine, trialkylphosphines, or triarylphosphines.

The ring-opening reaction to form the halohydrin intermediate can be performed at a temperature from about 60 to about 125° C. The reaction can be considered complete when the acid number was less than about 10 mg KOH/g, preferably less than about 5 mg KOH/g. The reaction mixture can then be cooled to around ambient temperature. It is an advantage of the disclosed methods that isolation of the halohydrin intermediate is not necessary prior to the ring-closing reaction.

The disclosed methods include a ring-closing reaction to convert the halohydrin intermediate to the glycidyl ester. Ring-closure can be accomplished by combining the reaction mixture including the halohydrin intermediate with a basic solution comprising an alkaline base, water, optionally a water-soluble organic solvent, and optionally an organic solvent, to form a reaction mixture, and allowing the reaction to proceed at a temperature of up to about 30° C. to provide the glycidyl ester. In some embodiments, the temperature of the ring-closing step is maintained up to about 25° C., or up to about 20° C., or up to about 15° C., or up to about 10° C..

The ring-closing reaction can be performed at around ambient temperature to form the glycidyl ester. Conventional methods can require elevated temperatures (e.g., 60-80° C.), leading to side-product formation. Surprisingly and unexpectedly, the inventors hereof discovered that quantitative conversion of the halohydrin intermediate to the glycidyl ester could be achieved by performing the ring-closing step at a temperature up to about 30° C. under basic conditions while minimizing the amount of side-product formation. A basic solution of an alkaline base, water, optionally a water-soluble organic solvent, and optionally an organic solvent is added at a rate such that the temperature is up to about 30° C. The inorganic base can include an alkaline base, an alkaline earth base, or a combination thereof. The inorganic base acts to neutralize the acid as it is generated in the reaction. Specifically, when the hydroxyl of the halohydrin intermediate attacks the adjacent halogenated carbon, hydrogen halogen (e.g., hydrogen chloride) is generated, which can be neutralized with the inorganic base. The amount of inorganic base added to the ring-closing reaction can be up to about 1.5 eq., or up to about 1.2 eq., based on the molar amount of epihalogenhydrin added to the reaction in step (a). Preferably the inorganic base is added in a stoichiometric amount (i.e., about 1 eq.), based on the molar amount of epihalogenhydrin added to the reaction in step (a). When present, water-soluble solvents include alcohol solvents, such as methanol, ethanol, and isopropanol, ether solvents, such as tetrahydrofuran, 1, 2-dimethoxy-ethane; acetonitrile; acetone; N,N-dimethylformamide; dimethyl sulfoxide; or any combination thereof. The amount of the water-soluble solvent added should be an amount from 0.5 to 5 times by weight of the halohydrin intermediate. In some embodiments, the first step is performed neat (i.e., in the absence of solvent). In such embodiments, the organic solvent is added to the halohydrin intermediate prior to the addition of the basic solution. The water-soluble solvent can be present from about 10 to about 50 wt%, from about 10 to about 40 wt%, from about 10 to about 30 wt%, or from 10 to about 25 wt%, each based on the sum of total weight of the halohydrin intermediate.

The disclosed methods provide quantitative conversion of the halohydrin intermediates to the glycidyl ester, wherein the presence of side-products is reduced or minimized in the glycidyl ester reaction product. As used herein, “glycidyl ester reaction product” refers to the crude product isolated after work-up of the reaction mixture of step (d). In some embodiments, the side-products comprise less than about 15%, less than about 10%, or less than about 5% of the glycidyl ester reaction product as determined by GPC.

The disclosed methods provide quantitative conversion of the halohydrin intermediates to the glycidyl ester reaction products having improved purity. In some embodiments, the glycidyl esters are at least 60 % pure, 65 % pure, 70% pure, 75 % pure, 80% pure, 85% pure, 90% pure, 95 % pure, 98 % pure, 99% pure, or 99.9 % pure as determined by GPC.

The disclosed methods advantageously provide improve percent yields of the glycidyl ester reaction product from the carboxylic acid substrates. Improved % yields are particularly desirable on an industrial scale. In certain embodiments, the % yield ranges from greater than 60%, greater than 70%, preferably greater than 80%.

The disclosed methods can further comprise reacting the glycidyl ester with a nucleophile in the presence of a ring-opening catalyst. The ring-opening catalyst can include an acid, a base, or a phosphine. The nucleophile can include water (OH), a hydroxyl functionalized amine, acrylic acid, methacrylic acid, a fatty acid, a thiol, an amine, or an alcohol. In some embodiments, a glycidyl ester can be converted to a diol by reaction with water in the presence of acid catalyst. In some embodiments, a glycidyl ester can be converted to a triol by reaction with diethanolamine in the presence of a triphenylphosphine catalyst. In some embodiments, a glycidyl ester can be converted to a methacrylate by reaction with glycidyl methacrylate.

In another aspect, the description provides a glycidyl ester prepared according to the methods described herein. In any of the aspects or embodiments, the glycidyl ester has the features as described herein.

The glycidyl esters obtained according to the methods described herein can be used to provide derivatives. Accordingly, in an additional aspect, the description provides rosin derivatives including a diol, a triol, an amine-containing product, and carboxylic acid reaction products.

### EXAMPLES

In the examples below, the acid number was measured by a Metrohm auto-titrator with KOH solution by ASTM D664. The epoxy equivalent weight (EEW) was determined by titration with perchloric acid in acetic acid.

Samples were analyzed by WATERS GPC equipped with a 2707 Autosampler and 2414 Refractive Index Detector. Data acquisition and handling were made with BREEZE 2 software.

Data were obtained under the following conditions:

| | |
|---|---|
| Solvent | THF |
| Flow Rate | 1.0 mL/min |
| Injection Volume | 25 µL |
| Column Temperature | 40° C. |
| Concentration | ~10 mg/mL |
| Column | 1× Ultrastyragel 500 Å (7.8×300 mm 10571 (100 - 10 K), 2× Ultrastyragel 100 Å (7.8×300 mm 10570 (100 - 5 K) |
| Run Time | 35 Minutes |

The details of the examples are contemplated as further embodiments of the described methods and compositions. Therefore, the details as set forth herein are hereby incorporated into the detailed description as alternative embodiments.

### Example 1. Synthesis of Epoxide of TOFA

A solution was prepared from the tall oil fatty acid (200 g, available as INGEVITY ALTAPYNE L5) and 1.5 g of tetrabutylammonium bromide (TBAB) in 200 mL of toluene. The solution was transferred to an addition funnel and added dropwise to epichlorohydrin (“ECH,” 79 g, 1.2 molar equivalent, based on TOFA) over 2 h at 110° C. The acid number was measured to monitor the reaction progression. After the acid number was below 4 mg KOH/g the halohydrin product was cooled to room temperature. For the ring closure, isopropyl alcohol (IPA, 47 g) was added, followed by the addition of aq. KOH (96 g, aq. 45 wt%) while maintaining the temperature below 30° C. The reaction proceeded for 30 mins. The reaction mixture was filtered and the filtrate was neutralized with aq. acid. The filtrate was extracted with organic solvent (such as toluene), washed with water and then brine. The organic layer was concentrated by evaporation to provide the crude product in an 85 % yield. Analysis by GPC indicated the formation of the product. The epoxy equivalent weight (EEW) was 460 as determined by titration with perchloric acid in acetic acid.

### Example 2. Epoxide of Disproportioned Resin

Disproportioned rosin (1212 g, ALTAPYNE 505) was charged to a 3 L flask and 800 ml toluene was used to dissolve the rosin. Then 3 g triphenylphosphine was added to catalyze the reaction. After that, ECH (414 g, 1.21 eq.) was added dropwise over 1 hour at 95° C. and the reaction proceeded for total of 8 hours. The reaction was monitored by GPC and acid value. When the acid number of reaction mixture was below 5 mg KOH/g, the reaction mixture was cooled to room temperature.

To the cooled reaction mixture was added aq. KOH (538 g, 45 wt%). The reaction mixture was diluted by 25% with DI water along with small amount of water-soluble organic solvent such as methanol and stirred at room temperature for 8 hours. The reaction was monitored by GPC and epoxy equivalent weight (EEW). The glycidyl ester product was obtained a quantitative yield having 80% purity as determined by GPC. The EEW of the product was 505, as determined by titration with perchloric acid in acetic acid.

### Example 3: Epoxidation of DTO

A solution of DTO (507 g, INGEVITY ALTAPYNE M-28B) and TBAB (4.0 g) in 300 mL of toluene was prepared, transferred to an addition funnel, and added dropwise to 245 g of ECH at 100° C. The acid number was measured to monitor the reaction progression. After the acid number was below 1 mg KOH/g the halohydrin intermediate was cooled to room temperature. Then, IPA (159 g) was added, followed by the addition of aq. KOH (324 g, 45 wt%) while maintaining the temperature below 30° C. The reaction proceeded for 30 mins. The reaction mixture was filtered and the filtrate was neutralized with aq. acid. The filtrate was extracted with toluene, washed sequentially with water and then brine. The organic layer was concentrated by evaporation to provide the crude glycidyl ester product in an 84 % yield. Analysis by GPC indicated the formation of the product. The EEW was 660, as determined by titration with perchloric acid in acetic acid.

### Example 4: Diepoxide of Diacid 1550

A solution of (212 g, INGEVITY Diacid 1550) and TBAB (3.0 g) in 200 mL of toluene was prepared, transferred to an addition funnel, and added dropwise to ECH (142 g, 1.2 molar equivalent per carboxylic acid group) at 110° C. The acid number was measured to monitor the reaction progression. After the acid number was below 1 mg KOH/g the halohydrin intermediate was cooled down to room temperature. Then, IPA (92 g) was added to the halohydrin intermediate reaction mixture and stirred thoroughly. It was followed by the addition of aq. KOH (187 g, 45 wt%) while maintaining the temperature below 30° C. The reaction proceeded for 30 mins and the reaction mixture was filtered. The filtrate was neutralized with aq. acid and extracted with toluene. The organic layer was washed sequentially with water and then brine and dried over MgSO₄. After filtration, the filtrate was concentrated by solvent evaporation. The yield of epoxide product was 87 %. Analysis by GPC indicated the formation of the product. The EEW was 410.

### Example 5: Triepoxide of Fumarated TOFA

Fumarated TOFA (166 g, INDULIN 201) and TBAB (0.4 g) was mixed with 80 mL toluene and ECH (107 g, 1.16 eq. per carboxylic acid group) was added slowly. The solution was heated to 95° C. then 100° C. for 6 hours. The acid number was measured to monitor the reaction progression. After the acid number was 3 mg KOH/g, the halohydrin intermediate reaction mixture was cooled to room temperature.

Aq. KOH (85 g, 45 wt%), IPA (25 g), and DI water (45 g) were added to the reaction mixture. After 1 hour, additional aq. KOH (31 g, 45 wt%) was added and mixed for 30 min. The bottom aqueous layer was removed, the top layer was washed with water (½ organic/water) and allowed to settle overnight. The organic layer was allowed to sit overnight. The solvent was removed from the organic layer to provide the epoxide product in an 85% yield having 75% purity by GPC. The EEW was 478.

### Example 6: Triepoxide of Fumarated Rosin Acid

Fumarated rosin acid (1279 g Stafor size adduct) was added to a 3L flask, then 630 g toluene was added to dissolve the product. The acid number was measured 252.5 mg KOH/g. ECH (620 g, 1.2 eq. per carboxylic acid group) was added dropwise over 2 h at 90° C. The reaction was then stirred at 100° C. for 8 hours. When acid number was 5 mg KOH/g the reaction mixture was cooled to room temperature. Then KOH pellets (375 g) were added, followed by the addition of 200 g of water, and the reaction proceeded at room temperature for 8 hours. The bottom aqueous layer was removed, the top layer was washed with water (½ organic/water) and allowed to settle overnight. The organic layer was allowed to sit overnight. The organic layer was then vacuum stripped to remove solvent to provide an 80% yield, having a purity of 65% by GPC. The EEW was 455.

### Example 7: Epoxide of Dimer Acid

Rosin dimer (74 g, ALTAPYNE 595) with an acid number of 226 was charged to a 500 mL flask and ECH (41 g, 1.5 eq. per carboxylic acid group) was added along with 100 mL toluene and 0.35 g TBAB as catalyst and heated to 105° C. for 6 hours. The acid number was measured to monitor the reaction progression. The acid number was 5 mg KOH/g after 10 hours and cooled to room temperature. Aq. KOH (55 g, 45 wt%) was added along with 20 g DI water and 25 g isopropanol. The reaction mixture was stirred at room temperature for 2 hours and according to GPC, the reaction was complete. The bottom aqueous layer was removed and the top layer was washed with water (½ org /water) and allowed to settle overnight. The organic layer was then concentrated to remove the solvent to provide an 80% yield having a purity of about 75% by GPC. The EEW was 556.

### Example 8: Ring-Opening of Rosin Epoxide With Water

To the rosin epoxide from Example 2 (850 g) was added 400 mL toluene, 200 mL methanol, 200 g water, and 30 g concentrated H₂SO₄. The reaction mixture was heated to 75° C. for 1 hour. GPC analysis showed that all of the epoxy groups had been converted to diol. The crude product was washed with 15 g NaHCO₃ in 200 mL water and the organic layer was concentrated to remove the solvent and the nitrogen-sparged to remove residual solvent and water. The diol product was obtained in a 90% yield.

### Example 9: Ring-Opening of Rosin Epoxide With Diethanolamine

To the rosin epoxide (452 g, Example 2) was added diethanolamine (98 g) with 0.2 g triphenylphosphine as catalyst. The reaction mixture was heated at 90° C. for 2 hours until complete conversion of the epoxide to the DTO triol. No additional separation process was carried out. Quantitative yield was achieved.

### Example 10: Ring-Opening of Rosin Epoxide With Methacrylate

The rosin epoxide (631 g, Example 2) was added to a 1000 mL round bottom flask and heated it up to 135° C. When all of the DTO epoxide was melted, the flask was cooled to 125° C. and CYANOX 1790 (0.64 g) was added to the DTO epoxide. Then glycidyl methacrylate (302 g (GMA) was added to help solubilize the solid. Then, 0.6 g triphenylphosphine was added as catalyst. After 4 hours, the acid number was 9 mg KOH/g and reaction was stopped. No additional separation process was carried out. A quantitative yield was obtained.

### Comparative Example 11: Synthesis of a Glycidyl Ester of Rosin Acid

To 80% Altapyne 895 (aq., 1020 g, 2.2 mole) was added ECH (600 g, 6.5 mol), TBAB (5 g), and DI water (600 mL). The reaction mixture was heated to 80° C. The reaction progress was monitored by GPC/FTIR. After 12 hours, KOH pellets (85 g) were added along with 200 mL water and heated to 80° C. for 4 hours. The reaction was monitored by GPC until all of the ECH was consumed. After cooling to room temperature, the organic layer was removed and concentrated to provide the product. The product was 60% pure, with the rosin dimer and other impurities remaining. The EEW was 578.

While several embodiments of the invention have been shown and described herein, it will be understood that such embodiments are provided by way of example only. Numerous variations, changes and substitutions will occur to those skilled in the art without departing from the spirit of the invention. Rather, the present disclosure is to cover all modifications, equivalents, and alternatives falling within the scope of the present disclosure as defined by the following appended claims and their legal equivalents. Accordingly, it is intended that the description and appended claims cover all such variations as fall within the spirit and scope of the invention.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients can be varied to optimize the desired effects, additional ingredients can be added, and/or similar ingredients can be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

What is claimed is:

1. A method for the preparation of a glycidyl ester, the method comprising the steps of
a. admixing (i) a carboxylic acid substrate comprising a rosin acid or derivative thereof, a fatty acid or derivative thereof, or a combination thereof, (ii) a catalyst, and (iii) a molar excess of an epihalogenhydrin based on the total moles of carboxylic acid groups;
b. heating the reaction mixture at a temperature from about 60° C. to about 125° C. to form a halohydrin intermediate;
c. combining the reaction mixture comprising the halohydrin intermediate from step (b) with a basic solution comprising an inorganic base, and water; and
d. allowing the reaction to proceed at a temperature of up to about 30° C. to provide the glycidyl ester.

2. The method of claim 1, wherein step (d) is performed at ambient temperature.

3. The method of claim 1, wherein the halohydrin intermediate is not isolated prior to step (c).

4. The method of claim 1, wherein (i) the reaction mixture of step (a) further comprises an organic solvent or (ii) the reaction mixture of step (c) further comprises an organic solvent.

5. The method of claim 1, wherein the reaction mixture of (c) further comprises a water-soluble organic solvent.

6. The method of claim 1, further comprising step (e), wherein step (e) comprises isolating a glycidyl ester reaction product from the reaction mixture of step (d).

7. The method of claim 1, further comprising step (f), wherein step (f) comprises reacting the glycidyl ester with a nucleophile in the presence of a ring-opening catalyst to provide a ring-opened derivative.

8. The method of claim 7, wherein the nucleophile comprises water, a hydroxyl functionalized amine, acrylic acid, methacrylic acid, a fatty acid, a thiol, an amine, or an alcohol.

9. The method of claim 7, wherein the ring-opening catalyst comprises an acid, a base, or a phosphine.

10. The method of claim 1, wherein the rosin acid derivative or the fatty acid derivative comprises one carboxylic acid group, two carboxylic acid groups, or three carboxylic acid groups.

11. The method of claim 1, wherein the rosin acid derivative comprises a disproportionated rosin, a maleated rosin, a fumarated rosin, an acrylonitrile adduct, itaconic acid adduct, an acrylic acid adduct, a dimer acid, an oxidized rosin, a hydrogenated rosin, or a combination thereof.

12. The method of claim 1, wherein the fatty acid derivative comprises a maleated fatty acid, an acrylonitrile fatty acid adduct, a fumarated fatty acid adduct, acrylic-acid fatty acid adduct, an itaconic acid fatty acid adduct, a dimer fatty acid, an oxidized fatty acid, a hydrogenated rosin acid, or a combination thereof.

13. The method of claim 1, wherein the molar excess of epihalogenhydrin is from greater than about 1 to about 2, based on the moles of carboxylic acid groups present in the substrate.

14. The method of claim 4, wherein the organic solvent comprises toluene, xylene, hexane, heptane, or a combination thereof.

15. The method of claim 1, wherein the catalyst comprises a phosphine, a tertiary amine, a quaternary ammonium salt, an onium salt, or a combination thereof.

16. The method of claim 5, wherein the water-soluble organic solvent comprises acetone, methanol, ethanol, isopropanol, acetonitrile, tetrahydrofuran, dimethylformamide, dimethyl sulfoxide, or a combination thereof.

17. The method of claim 1, wherein the method does not comprise a step comprising recovering excess epihalogenhydrin.

18. The method of claim 1, wherein the carboxylic acid substrate is derived from tall oil fatty acids, tall oil rosin, distilled tall oil, gum tree rosin, wood rosin, softwood rosin, hardwood rosin, a natural oil, or a combination thereof.

19. The method of claim 18, wherein the natural oil comprises vegetable oil, safflower oil, sesame oil, canola oil, olive oil, oil, coconut oil, or a combination thereof.

20. The method of claim 1, wherein a side-product comprises less than 15% of a glycidyl ester reaction product isolated from the reaction mixture of step (d), as measured by gel permeation chromatography.
